(19) United States
(12) Patent Application Publication
(10) Pub. No.: US 2008/0249005 A1
(43) Pub. Date: Oct. 9, 2008

(54) Use of Dm43 and Its Fragments as Matrix Metalloproteinases Inhibitor

(76) Inventors: Patricia Barbosa Jurgilas, Rio de Janiero (BR); Juliana de Meis, Rio de Janiero (BR); Richard Hemmi Valente, Rio de Janiero (BR); Ana Gisele da Costa Neves Ferreira, Rio de Janiero (BR); Daniella Areas Medes da Cruz, Rio de Janiero (BR); Desio Aurelio Farias de Oliveira, Rio de Janiero (BR); Wilson Savino, Rio de Janiero (BR); Gilberto Barboss Domont, Rio de Janiero (BR); Jonas Enrique Perales Aguilar, Rio de Janiero (BR)

Correspondence Address: KLARQUIST SPARKMAN, LLP, 121 SW SALMON STREET, SUITE 1600, PORTLAND, OR, 97204, US

(21) Appl. No.: 10/593,527

(22) PCT Filed: Mar. 18, 2005
(86) PCT No.: PCT/BR05/00036

§ 371 Date: Jul. 25, 2007

(30) Foreign Application Priority Data
Mar. 18, 2004 (BR) 0400284-9

(51) Int. Cl. A61K 38/16; A61P 25/28; A61P 19/02; A61P 35/00

(52) U.S. Cl. 514/12

(57) ABSTRACT

This invention is related to the use of DM43 protein as a metalloproteinases inhibitor. More specifically, this invention is related to the use of DM43 for the therapeutics of pathologies such as cancer and osteoarthritis, which are dependent on matrix metalloproteinases inhibition, based on the structure identity with SVMPs.

### FIELD OF THE INVENTION

This invention, in its broader concept, refers to the use of DM43 protein as a metalloproteinases inhibitor. More specifically, this invention is related to the use of DM43 for therapeutic treatment in matrix metalloproteinases inhibition, and a pharmaceutical composition consisting of DM43 protein plus a pharmaceutically acceptable vehicle.

### RATIONALE OF THE INVENTION

DM43 protein is a well-known molecule which has been purified and characterized as a snake venom metalloproteinases inhibitor. However, the technique status has not presented reports of the ability of DM43 to modulate cellular functions such as apoptosis induction, increased deposit of extracellular matrix components and increased cellular adhesion.

The inventors in this patent application have been investigating the natural resistance of marsupials to snake venom for many years. A 43 kDa acid glycoprotein (DM43) which can inhibit the activities of metalloproteinases encountered in snake venom (SVMPs) [Snake Venom Metalloproteinases] through the formation of noncovalent complexes has been isolated and characterized from opossum (Didelphis marsupialis) serum [(Neves-Ferreira et al., 2000) and (Neves-Ferreira et al., 2002)].

The SVMPs belong to the family of Metzincins, whose members have common characteristics according to their topology, zinc-binding consensus sequence and a conserved methionine residue, which forms the basis of active sites. They share approximately 15% of structure identity with matrix metalloproteinases (MMPs), and 30% with disintegrin-like domain metalloproteinases (ADAMSs). (Nagase and Woessner, 1999)

The MMPs are currently described as a family of 26 members, which stand out for their participation in various physiological (fetal development and immune system regulation) and pathological processes. The most important regulation mechanism of metalloproteinases activity is the formation of a complex with their natural inhibitors, the TIMPs (tissue inhibitors of metalloproteinases). The disequilibrium between TIMPs and MMPs may result in the most diverse pathologies, such as articular diseases, cancer, cardiovascular diseases, neurological disorders, etc.

The most common articular disease among humans is osteoarthritis, which attacks about 190 million people throughout the world, according to the World Health Organization. The pathogenesis is a consequence of the progressive degeneration of extracellular matrix components of diarthroses articular cartilages, as a result of the proteolytic action of MMPs. These metalloproteinases reach high levels in animal models, in human cartilages and in the synovial liquid of osteoarthritis patients (Janusz et al., 2002).

Current therapeutics for osteoarthitis patients consists of using anti-inflammatories, which are only aimed at pain relief and minimization of motor disability. There have been studies aimed at MMPs inhibition or block for the treatment of this disease. Among these, MMP-3 has been referred to as the main molecular target, since it is an enzyme which acts on a great deal of extracelullar matrix proteins, also having the ability of activating other MMPs. For being overexpressed in this physiopathologic process, it is used as a disease marker (Lohmander et al., 1993).

Over the last thirty years, the MMPs have been considered promissing targets in cancer therapy. This theory has been supported by an increased genic expression of these metalloproteinases in malignant tissues and by their ability to degrade extracellular matrix components. In several cases, tumor progression is related to the expression levels of MMPs. Alterations in these levels may also evidence the invasive behavior of tumor cells, and their ability to provoke metastasis in experiments with animal models (Coussens et al., 2002). There have been studies focusing on the inhibition of these metalloproteinases activity through the use of their natural inhibitors, the TIMPs, or the use of synthetic compounds which might inactivate these enzymes. Moreover, the possibility of MMPs synthesis inhibition has been studied (Vincenti et al., 1994). Several groups have already demonstrated that the overexpression of TIMPs may reduce experimental metastasis. Nevertheless, technical difficulties have prevented the use of TIMPs as potential drugs so far.

The use of synthetic inhibitors has been tested in the pharmaceutical industry, with no relevant results up to now. Besides presenting low bioaccessibility, they may give rise to pain and severe inflammations as side effects. The state of the art (Coussens et al., 2002, table of this article) has revealed that (Phase III) clinical trials with known inhibitors have not shown relevant results up to the moment.

Thus, the discovery of a new matrix metalloproteinases inhibitor may mean a therapeutic advance for pathologies with no treatment perspective at present.

### SUMMARY OF THE INVENTION

The main goal of this invention is the therapeutic use of DM43 protein as an inhibitor of matrix metalloproteinases involved in pathologies such as cancer and osteoarthritis.

### BRIEF FIGURES DESCRIPTION

FIG. 1 shows cell viability in normal murine fibroblasts (3T3).

FIG. 2 shows cell cycle in normal murine fibroblasts (3T3).

FIG. 3 shows apoptosis in normal murine fibroblasts (3T3).

FIGS. 4A, 4B and 4C show cell viability, cell cycle and apoptosis with murine tumor cell line (EL4), respectively.

FIGS. 5A, 5B, 5C and 5D show extracellular matrix components labeling (fibronectin) in normal murine fibroblasts (3T3): negative control and 3 different DM43 concentrations.

FIGS. 6A, 6B, 6C and 6D show cell adhesion in normal murine fibroblasts cell lines (3T3) in a coculture with thymocytes of the negative control and of 3 different DM43 concentrations.

FIG. 7 shows the supernatant profile of normal murine fibroblasts (3T3) when subjected to DM43-coupled affinity chromatography.

FIG. 8 shows silver-impregnated (12.5%) polyacrylamide gel electrophoresis, where numeral 1 accounts for the molecular mass standard, numeral 2 accounts for the fibroblasts supernatant and numeral 3 accounts for the fraction bound to affinity chromatography.

FIGS. 9A and 9B show prostate (MDA) and breast (MCF7) adenocarcinoma supernatant profiles, respectively, when subjected to DM43-coupled affinity chromatography.

FIG. 10 shows silver-impregnated (12.5%) polyacrylamide gel electrophoresis, where numeral 1 accounts for the molecular mass standard, numeral 2 accounts for MDA supernatant, numeral 3 accounts for the MDA fraction which is not bound to affinity chromatography, numeral 4 accounts for the MDA fraction which is bound to affinity chromatography, 5 accounts for empty space, numeral 6 accounts for MCF7 supernatant, numeral 7 accounts for the MCF7 fraction which is not bound to affinity chromatography and numeral 8 accounts for the MCF7 fraction which is bound to affinity chromatography.

FIG. 11 shows 10% SDS-PAGE acrylamide gel zymography of osteoarthritis synovial liquid.

FIG. 12 shows the inhibition assay result using casein as substrate, where osteo accounts for osteoarthritis synovial liquid, serum accounts for opossum serum and O+serum accounts for the osteoarthritis synovial liquid incubated with opossum total serum over 30 minutes at 37° C.

FIG. 13 shows the osteoarthritis synovial profile when subjected to DM43-coupled affinity chromatography.

FIG. 14A shows silver-impregnated polyacrylamide gel electrophoresis of osteoarthritis synovial liquid, where numeral 1 accounts for molecular mass standard, numeral 2 accounts for synovial liquid, numeral 3 accounts for the affinity ligand, numeral 4 accounts for empty space and numeral 5 accounts for BSA.

FIG. 14B shows the immunotransference of nonreduced samples revealed with anti-MMP-3 monoclonal antibody, [sic], where numeral 1 accounts for pre-stained MS) [sic] pre-stained molecular mass standard, numeral 2 accounts for synovial liquid, numeral 3 accounts for the affinity ligand, numeral 4 accounts for BSA, as negative control.

FIG. 15 shows the molecular mass of diverse MMPs which have already been described in the literature.

FIGS. 16A and 16B show DM43-coupled affinity chromatography of prostate adenocarcinoma supernatant and breast adenocarcinoma supernatant, respectively.

FIG. 17 shows polyacrylamide gel zymography.

FIGS. 18A, 18B and 18C represent the immunorevelation with anti-MMPs monoclonal antibody.

FIGS. 19A and 19B show fibronectin degradation and the fibronectin degradation inhibition scheme, respectively.

FIG. 20 shows the MDA cells number analysis.

FIG. 21 shows the MCF7 cells number analysis.

FIG. 22 shows the MDA cell cycle analysis.

FIG. 23 shows the MCF7 cell cycle analysis.

FIGS. 24A and 24B show the MDA cell death analysis.

FIGS. 25A and 25B show the MCF7 cell death analysis.

FIG. 26 shows the adhesion and block of thymocytes over fibroblasts.

### DETAILED DESCRIPTION OF THE INVENTION

The invention will now be described through the examples below, which should not be interpreted in a restrictive manner.

In the present invention, the murine fibroblasts models (3T3), murine lymphomas (EL4), human adenocarcinomas (breast—MCF7 and prostate—MDA) and osteoarthritis have been selected on the basis of the following aspects:

(i) murine fibroblasts: main normal cell line producer of MMPs;

(ii) murine thymus lymphoma (EL4): thymus tumor cell line, the literature reveals that tumor cell lines overexpress MMPs;

(iii) human adenocarcinomas (breast and prostate), tumor cell lines overexpress MMPs, mainly MMPs-2 and MMPs-9 in such cases;

(iv) osteoarthritis: main articular disease in humans, in which pathogenesis is given by progressive degradation of extracellular matrix components of diarthroses cartilages by MMPs.

### EXAMPLE 1

### Obtention of DM43 Protein

DM43 protein has been obtained according to a method described by (Neves-Ferreira et al., 2000) and (Neves-Ferreira et al., 2002), incorporated here for reference.

The serum obtained from Didelphis marsupialis, captured in the State of Rio de Janeiro in accordance with the standards recommended by the IBAMA, was subjected to dialysis under agitation over 24 hours at 4° C. against 0.01 M sodium acetate buffer, pH 3.7, and centrifugated at 7,800×g (BECKAN J2-21M refrigerated centrifuge) for 15 minutes at 10° C. The supernatant was fractionated by (2.5×30 cm) DEAE-Sephacel gel anion-exchange chromatography. The elution was initially carried out isocratically, followed by a gradient of 0.15-0.5 M NaCl in the same dialysis buffer, at a flow rate of 30 mL/hour. The active fraction was precipitated with ammonium sulfate (80% of saturation) and subjected to (1.6×20 cm) Phenyl Sepharose gel hydrophobic interaction chromatography. The fractionating was carried out using a linear gradient of 0.5 M of ammonium sulfate to the buffer (0.1 M sodium phosphate, pH 7.0) and the same buffer without sulfate addition, at a flow rate of 30 mL/hour. All the chromatographies were performed in a Pharmacia Biotech AKTA purifier system. DM43 was dialysed against ammonium carbonate and lyophilized.

### EXAMPLE 2

### Obtention of Normal Cell Lines, Tumor Cell Lines and Synovial Liquid

The mouse fibroblasts cell line (3T3) was cultured in DMEM (Dulbecco's Modified Eagle's Medium) added with 10% fetal serum, 5% CO₂ at 37° C.

Prostate adenocarcinoma cell lines (MDA), as well as those of breast adenocarcinoma (MCF7) were provided by the Instituto Nacional do Cancer [National Cancer Institute]—INCA.

The osteoarthritis synovial liquid was provided by the Hospital Universita rio Clementino Fraga Filho [Clementino Fraga Filho University Hospital](UFRJ).

### EXAMPLE 3

### Proteins Ddosage

Protein content determination of the samples was carried out in accordance with the method of (Lowry et al., 1951). The standard curve was built from a 1 mg/mL solution of bovine albumin serum (BSA), with points of 10-50 μg.

### EXAMPLE 4

### Cell Viability

The fibroblasts cell line (1×10⁵ cells/bottle) was incubated in DMEM medium at 37° C. After 24 hours, the cells were [sic] and challenged with 3 different concentrations of DM43 (10, 250 and 1000 ng/mL) over 20 hours at 5% CO₂, 37° C. The viability analysis was performed by cell exclusion using Trypan blue stain in a Neubauer chamber. This staining evidences cells which are in process of cell death. Thus, in this experiment, we have only counted non-stained, and therefore, viable cells.

### EXAMPLE 5

### Cell Cycle

After 24 hours of incubation, as described above, the fibroblasts (1×10⁵ cells/bottle) were challenged for 20 hours with 10, 250 and 1000 ng/mL of DM43 at 5% CO₂, 37° C. The cell cycle was assessed using 1 mL of Vindelov O stain (Sigma Co. St. Louis, USA), composed of 3.4 mM tris-HCl, pH 7.6, 0.75 mM propidium iodide (PI), NP-40 (v/v %, 0.1), 700 UL bovine pancreas ribonuclease and 10 mM NaCl (Vindelov and Christensen, 1990). After 20 minutes at 4° C., the cells and [sic] were analyzed in FACScalibur® flow cytometry using the “software” Cell Quest. This stain evidences DNA and, depending on the cycle phase in which the cell is, greater or lesser fluorescence is observed, evidenced by the PI marker. The analysis of the results was performed by (area and width) [sic], following the acquisition in linear scale. It is worth emphasizing that 15000 cells were obtained regardless of size (FSC) and granulosity (SSC) predetermined parameters.

### EXAMPLE 6

### Cell Death

The fibroblasts were kept in culture (37° C., 5% CO₂) over 24 hours in DMEM medium, and challenged with (10; 250; 1000 ng/mL of DM43) [sic] for 20 hours. After this period, they were incubated with 20 mg/mL of the marker 7-actinomycin D (7AAD) for 20 minutes and analyzed by flow cytometry)[sic] (Philpott et al., 1996), as described previously. This reagent evidences DNA in a differential manner. Viable cells are not evidenced, once they are intact. Necrosed cells are the most evidenced by the stain, once their plasma membranes have been ruptured; apoptotic cells are poorly evidenced and, as well as necrotic cells, are smaller in comparison with viable cells. Thus, we can plot a chart with 3 different regions depending on the study cell status and cell size.

The same series of experiments (cell viability, cycle and death) was carried out using the EL4 tumor cell line (thymus lymphoma).

### EXAMPLE 7

### Extracellular Matrix Components Labeling

The experiments of extracellular matrix were performed using the same fibroblasts cell line, which was incubated with the same DM43 concentrations (10, 250 and 1000 ng/mL) for 20 h. After this period, the cells were labeled with anti-laminin primary antibody, fibronectin and collagen. After 30 minutes, the cells were washed and labeled with secondary antibody with fluorescein. The cells were analyzed by fluorescence optical microscopy. We have used bone marrow stroma cell line (S17) as positive control. As negative control, we have used a normal rabbit Ig. Anti-fibronectin and anti-laminin antibodies were used in the dilution of 1:100, whereas the anti-collagen was used in the concentration of 1:50.

### EXAMPLE 8

### Cell Adhesion

Cell adhesion was performed in fibroblasts cell line (1×10⁵ cells/well) plated for 24 h in an incubator at 37° C. and 5% CO₂. After that, the cells were challenged with the inhibitor (10, 250 and 1000 ng/mL) over 20 h. After this period, fresh thymocytes isolated from Balb mice were added to the culture (about 50 thymocytes per fibroblast). The coculture remained in the incubator at 37° C. and 5% CO₂ over 30 minutes, and was subsequently subjected to agitation for additional 30 minutes. Then, thymocytes which did not adhere or which were just weakly adhered were withdrawn through the inclination of the plate. Remaining cells were fixed with absolute methanol (MeOH) for 7 minutes and stained with Gimsa [sic] stain (dyes only the nuclei of the cells) for 30 minutes. The cells were then washed with distilled water and analyzed by optical microscopy. To perform the adhesion total analysis, the thymocytes which had not adhered or which had been weakly adhered were withdrawn by plate inclination, and the adhered ones were washed and counted in a Neubauer chamber.

### EXAMPLE 9

### DM43-Coupled Affinity Chromatography

DM43 inhibitor, which had been isolated as per Example 1, was coupled covalently to a 1 mL HiTrap® NHS affinity column (Amersham Biosciences), according to the instructions manual. The different materials (supernatants of fibroblasts, breast and prostate adenocarcinomas, and osteoarthritis synovial liquid) were precipitated with ammonium sulfate (80% of saturation) and subjected to the column, which was equilibrated with 0.02 M tris-HCl buffer+0.02 M CaCl₂, pH 7.5. Bound fractions were eluted with 0.1 M glycine-HCl buffer+0.02 M CaCl₂, pH 2.7, at a flow rate of 1 mL/min at 4° C., and collected into a 1 M tris solution in order to neutralize the pH of eluted fractions.

### EXAMPLE 10

### Polyacrylamide Gel Electrophoresis and Zymography

All the samples and eluted fractions were analyzed by SDS polyacrylamide gel electrophoresis, according to the method of (Laemmli, 1970). Concentration gels were constituted of 4% bis-acrylamide and the running gels were used in the concentration of 12.5%. The sample buffer was used in the presence of the reducing agent (5% β-mercaptoethanol). As running buffer, we used 0.05 M tris-glycine, pH 8.3, with 0.1% SDS. All the samples were heated at 100° C. for 5 minutes before being applied to the gel. We used the Mini Protean II (BIo RAD) system. The runs lasted an average of 40 minutes, using a constant voltage of 200 V. The gels were impregnated by silver for the revelation of protein bands.

For the verification of the proteolytic activity of the fractions, the technique used was zymography, which was performed in 10% acrylamide gel copolymerized with 2% casein. The samples (11.2−44.8 μg protein/well of synovial liquid) were prepared in a buffer containing SDS and in the absence of reducing agent. The run had a total duration of 2 hours, using a constant current of 15 mA (for 1 hour) and 20 mA (1 hour). After the run, the gel was transferred to 50 mM tris HCl buffer, pH 7.6, containing 2.5% triton x-100 for 1 hour. After this time, the gel was transferred to 50 mM tris HCl lysis buffer, pH 7.8, containing 150 mM NaCl and 5 mM CaCl2 over 24 hours at 37° C., under agitation.

The gels were stained with 0.1% Coomassie blue R-250.

### EXAMPLE 11

### Immunorevelation

The samples were electrotransferred following polyacrylamide gel electrophoresis in the presence of SDS and reducing agent (5% β-mercaptoethanol) onto a PVDF membrane (Immobilon-P, 0.45 μM), using the Mini Trans-Blot (BIO RAD) system. On average, the transferences lasted 1 hour, using a constant voltage of 100 V. The revelation was carried out with rat-produced anti-MMP3 monoclonal antibody. The secondary antibody used was rat anti-IgG IgG, conjugated with peroxidase (R & D systems).

### EXAMPLE 12

### Inhibition of Proteolytic Activity

Following the confirmation of the proteolytic activity of osteoarthritis synovial liquid, inhibition assays were performed also using casein (1%) as substrate. Opossum total serum (2.8 mg) was incubated with the synovial liquid (8.4 mg) for 30 minutes at 37° C., before being added to casein (500 μL) containing 25 μL of 0.08 M CaCl₂ for 1 hour at 37° C. The reaction was interrupted with 5% TCA (500 μL), and the mixture was centrifugated for 15 minutes at 3000 rpm. The supernatant was analyzed by spectrophotometry (SHIMADZU) with 280 nm reading.

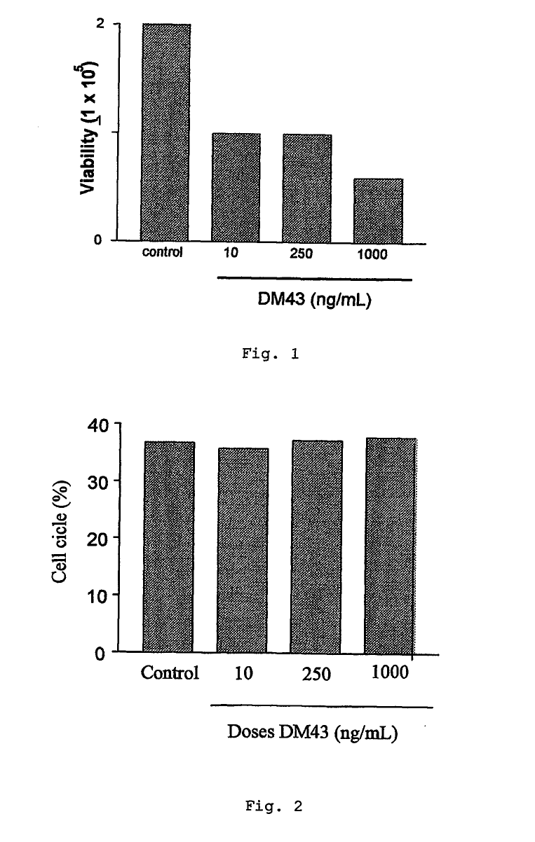

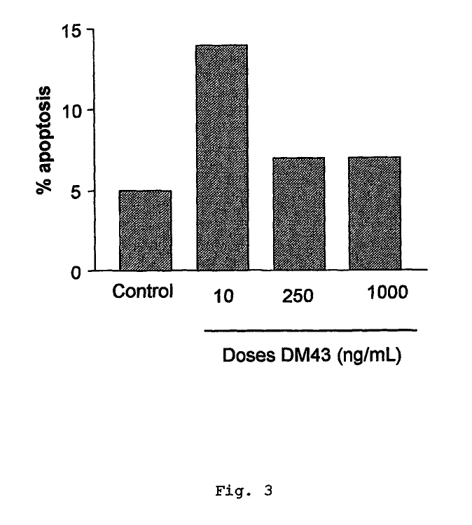

The results presented in the attached Figures demonstrate a decrease in fibroblasts viability using the 3 DM43 concentrations (10, 250 and 1000 ng/mL) (FIG. 1). This decrease could be caused by a cell death increase and/or cell cycle variation. In the fibroblasts cell cycle, we have not observed any significant effect with any of the treatment doses used (FIG. 2). However, the 7AAD analysis demonstrated increased apoptosis in the cells incubated with DM43, corroborating the data of cellularity. It is clear that the dose of 10 ng/mL is the most significant one within this phenomenon, trebling the number of apoptotic cells in relation to the control (FIG. 3).

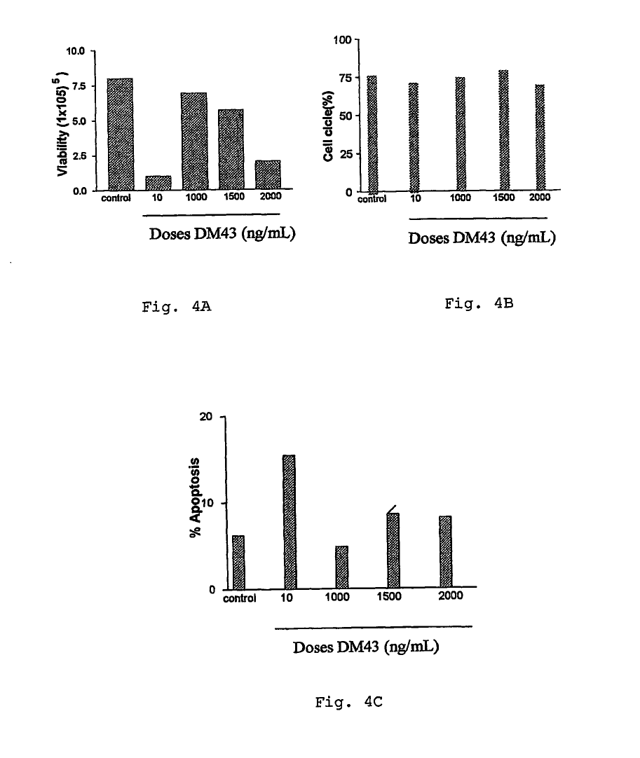

The results obtained with EL4 cell line showed the same bias of those obtained with fibroblasts, i.e., a decreased cell viability, caused by cell death, without demonstrating changes in the cycle of these cells (FIG. 4).

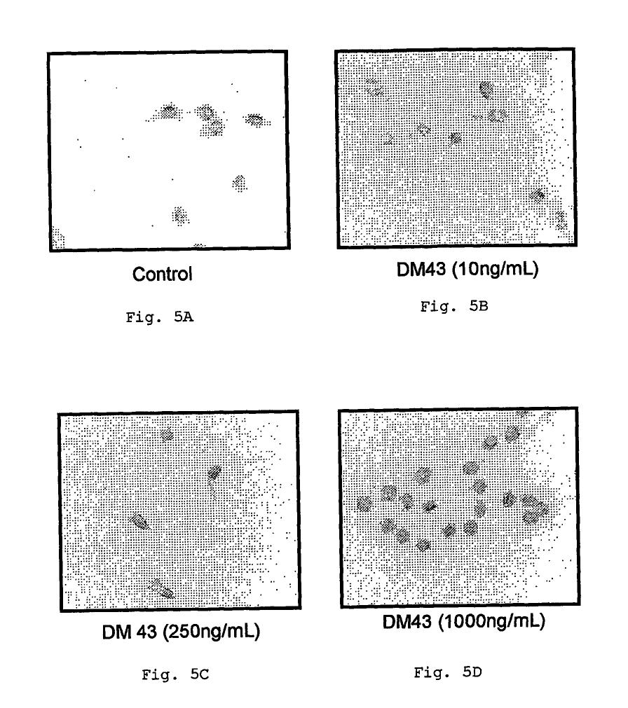

In the experiments of extracellular matrix components assessment, a great fibronectin deposit was verified, mainly with the use of a DM43 dose of 1000 ng/mL (FIG. 5). The other matrix components (laminin and collagen IV) have not demonstrated alterations.

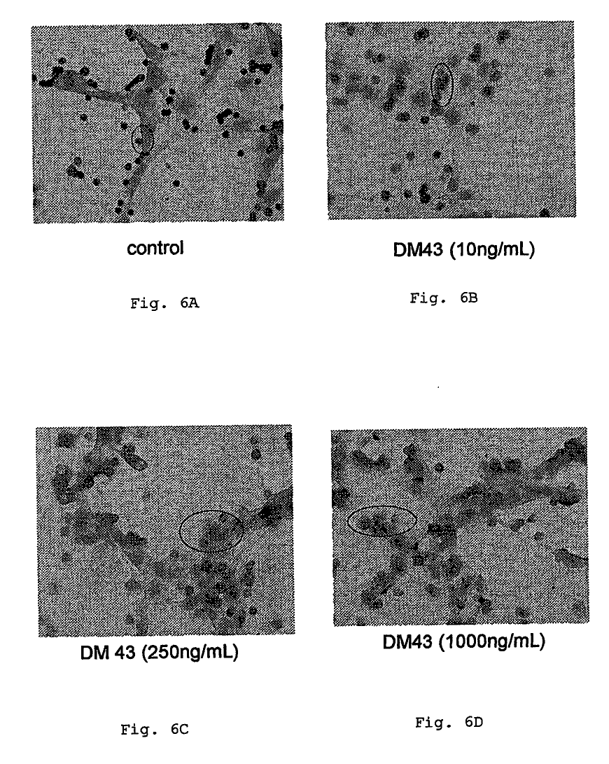

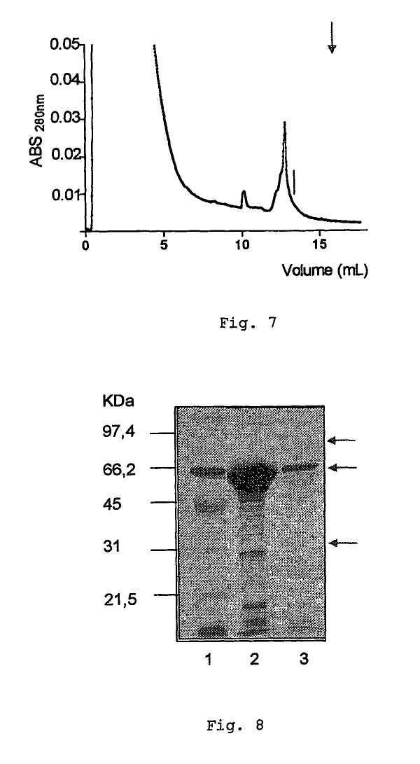

DM43 induced an increased adhesion, which seems to be dose-dependent, thus increasing according to the dose of inhibitor used (FIG. 6). Furthermore, a great development of thymocytes clots is observed, which made the quantification of these experiments impossible, as shown in FIG. 7.

TABLE 1
Cell Adhesion (fibroblasts/thymocytes)

| Adhesion | | DM43 | DM43 | DM43 |
|---|---|---|---|---|
| count | Control | (10 ng/mL) | (250 ng/mL) | (1000 ng/mL) |
| Total | 399 | 354 | “nd” | “nd” |
| fibroblasts | | | | |
| Thymocytes/ | 396 | 352 | “nd” | “nd” |
| fibroblasts | | | | |
| Total | 2116 | 2219 | “nd” | “nd” |
| thymocytes | | | | |
| Adhesion | 5.3% | 6.3% | “nd” | “nd” |
| index | | | | |

However, the total adhesion analysis revealed a significant increase using the doses of DM43, mainly that of 1000 ng/mL, as observed below in Table 2.

TABLE 2

| | | DM43 | DM43 | DM43 |
|---|---|---|---|---|
| | Control | (10 ng/mL) | (250 ng/mL) | (1000 ng/mL) |
| Total of | 7.5 × 10⁵ | 16.8 × 10⁵ | 15.3 × 10⁵ | 21.6 × 10⁵ |
| cells | | | | |

This increased adhesion of thymocytes to the fibroblasts and of the fibroblasts to the plate suggests an inhibition of metalloproteinases, which would be degrading matrix components, corroborating the data of increased fibronectin deposit, mainly with the highest dose used.

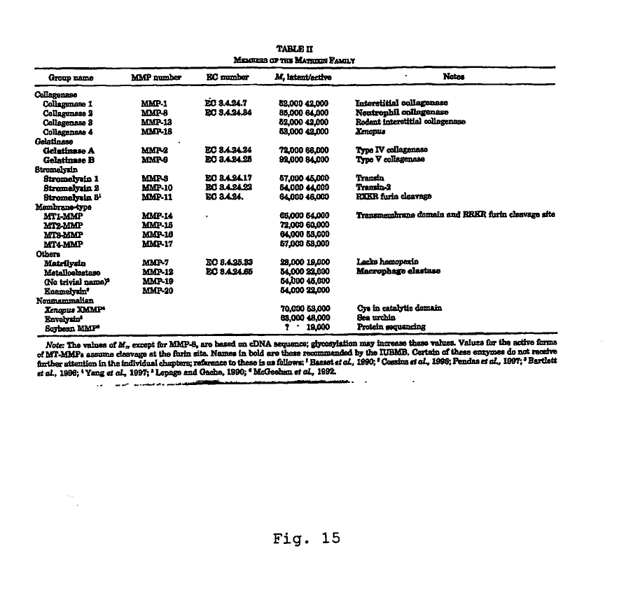

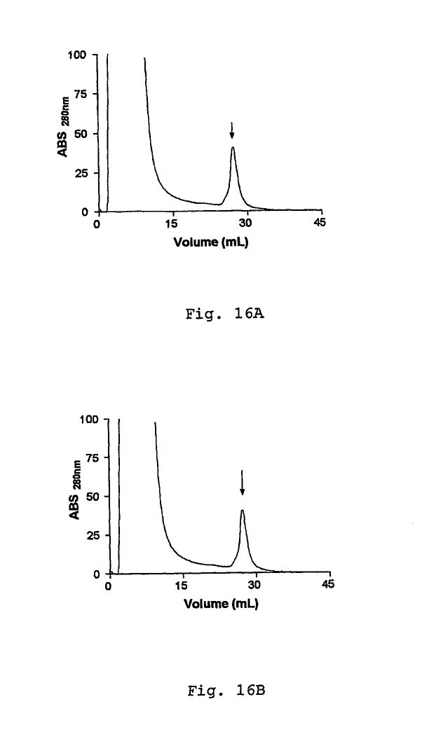

The results presented here demonstrated that a protein fraction of the fibroblast supernatant could interact with the DM43 coupled to NHS chromatography (FIG. 7). The analysis of this fraction by zymography indicates the presence of enzymes in its content, once it could hydrolyze the acrylamide-copolymerized casein. The electrophoretic analysis of this material demonstrates the presence of 3 main protein bands (FIG. 8). The molecular mass values of ≅92, 66 and 28 kDa corroborate with the molecular mass values reported for MMPs 2, 3 and 9, as shown in FIG. 15. MMP-3 may be in its active form with 28 kDa.

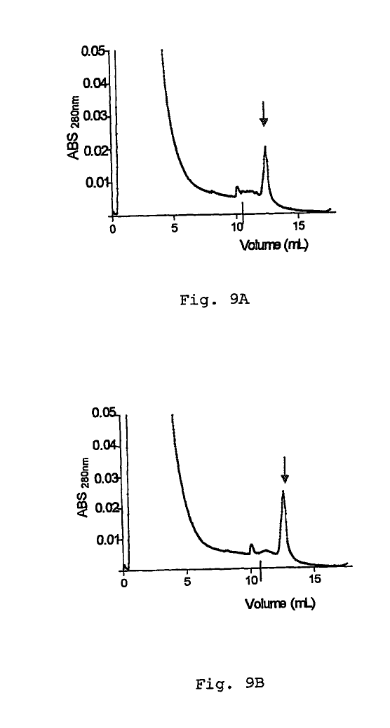

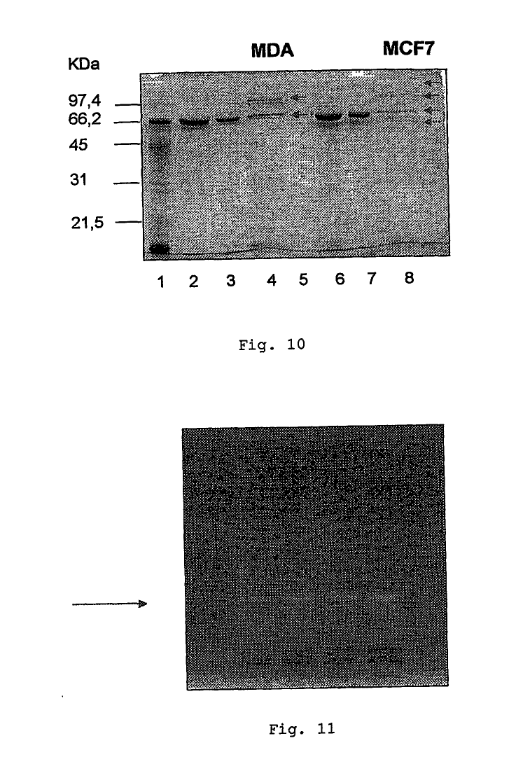

Similarly, protein fractions of the supernatants of tumor cell lines could bind to DM43 (FIG. 9) and hydrolyze casein. The electrophoretic analysis reveals the presence of 2 main protein bands of ≅92 and 66 kDa in the MDA bound fraction. As concerns MCF7, 4 protein bands of ≅98, 92, 66 and 55 kDa were visualized, as shown in FIG. 10. Once more, these mass values may suggest the presence of MMPs 2, 3 and 9.

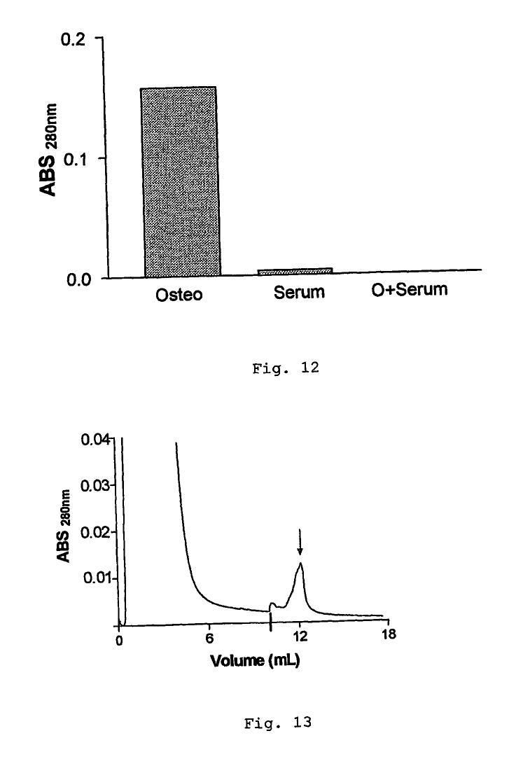

Following the confirmation of the proteolytic activity of the osteoarthritis synovial liquid by zymography (FIG. 11), this material was subjected to an inhibition assay using casein as substrate and, at 280 nm, it was verified that the liquid could hydrolyze casein, and that it was completely neutralized when incubated with opossum total serum (FIG. 12).

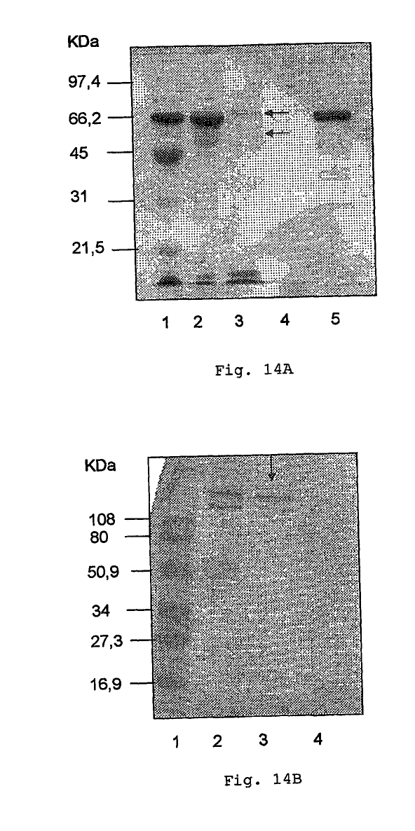

When the synovial liquid was subjected to affinity chromatography, a protein fraction binding was verified (FIG. 13). This fraction analyzed by SDS-PAGE revealed the presence of 2 main protein bands of ≅66 and 55 kDa (FIG. 14A). Anti-MMP-3 monoclonal antibody immunorevelation confirmed the presence of MMP-3 (FIG. 14B), indicating the interaction between DM43 and MMP-3 which was present in the osteoarthritis synovial liquid.

For the first time, this data indicates the interaction between a snake venom metalloproteinase inhibitor (DM43) and matrix metalloproteinases (MMPs). Moreover, the presence of protein bands of similar electrophoretic migration was verified in 4 different experimental models, which certainly correspond to MMP-2, 3 and 9.

In order to demonstrate the importance of DM43, new experiments were performed with this protein. New data resulting from assays of cellularity, cycle and apoptosis demonstrated that DM43 is also an apoptosis inducer in breast adenocarcinoma tumor cell line.

### EXAMPLE 13

### 1—Preparation of Cells and Treatment

All the cell lines (MDA and MCF7) were plated (1×10⁵ cells) in 25 cm² polystyrene bottles, in DMEM medium, and kept for 48 hours at 37° C. and 5% CO₂ before each experiment.

After this time, cells were treated with 3 different DM43 concentrations (10, 250 and 1000 ng/mL) for 20 hours within the conditions described above. After trypsinization and centrifugation at 8000×g for 5 minutes, the cells were analyzed as regards number, cycle and death.

2—Precipitation with TCA

The precipitation was conducted following the addition of 50% TCA (253 μL/mL of sample) and 1% Triton (127 μL/mL of sample) to the sample and incubation at 4° C. for 20 minutes. After that time, the sample was centrifugated at 16,000×g for 10 minutes. The supernatant was discarded, the protein precipitate was washed with cold acetone twice, centrifugated and dried at room temperature until complete acetone evaporation (Stone and Williams, 1993).

### 3—Zymography

The technique of zymography was used in order to verify the proteolytic activity of the fractions eluted with glycine/HCl buffer of the affinity chromatography, which was performed in 10% acrylamide gel copolymerized with 1% gelatin, as already demonstrated in example 10.

### 4—Immunorevelation

The samples were electrotransferred following polyacrylamide gel electrophoresis in the presence of SDS with or without reducing agent (5% β-mercaptoethanol), as described in example 11. The revelations were made with anti-MMP-2, anti-MMP-3 and anti-MMP-9 monoclonal antibodies produced in mice (R & D systems). The secondary antibody used was the rat anti-IgG IgG, conjugated with peroxidase (R & D systems).

### 5—Inhibition of the Proteolytic Activity Using Fibronectin as Substrate

Fibronectin (1 μg/μL) was incubated with breast and prostate adenocarcinoma fractions which interacted with DM43 immobilized in affinity chromatography. We evaluated the extension of hydrolysis with 0.5-15 μL of these fractions (1 mg/mL at 280 nm), at a period of 3 hours, at 25° C.

We selected the dose of 10 μL of the fractions containing the enzymes to test fibronectin degradation inhibition. Firstly, we incubated enzyme/DM43 mixtures (1:10 w:w) at 37° C. for 30 minutes. We also used orthophenanthroline (200 mM) in order to inhibit the enzymes. After this time, we added 3 μL of fibronectin (1 μg/μL) for 3 hours at 25° C. The reaction was interrupted with the addition of 5-fold concentrated Laemmli sample buffer in the presence of reducing agent. The analyses were performed through SDS-PAGE.

DM43 (100 μg), orthophenanthroline (200 mM) and fibronectin alone (3 μg) were used as controls at the same doses.

### 6—Cell Number Analysis

MDA and MCF7 cell lines were counted in a Neubauer chamber with the aid of an optical microscope.

### 7—Cell Cycle Analysis

MDA and MCF7 cell lines were analyzed according to the cycle, as mentioned in example 5.

### 8—Cell Death Analysis

For death analysis, MDA and MCF7 cell lines were washed with PBS and incubated with 20 mg/mL of marker 7-actinomycin D (7AAD) over 20 minutes and analyzed by flow cytometry (Philpott et al., 1996), as described previously in example 6.

Absolute viability was evaluated using the cellularity number and the percentage value reached through DOT PLOT. This number was subtracted from the total number of cells for us to have the viable cells, represented by a bar chart.

### 9—Cell Adhesion Block

The adhesion was performed in fibroblast cell lines treated with DM43 (10, 250 and 1000 ng) for 20 h, as already described in example 8.

The block was provided through the incubation of cells treated with anti-fibronectin monoclonal antibody over 30 minutes. After this interval, fresh thymocytes were added to the culture, following the same adhesion protocol (example 8).

For us to plot the adhesion index (AI), we proceeded the counts of total fibroblasts, total thymocytes and fibroblasts with adhered thymocytes. Counts involved about 500 cells per field.

$$AI = \frac{fibroblasts with adhered thymocytes}{total fibroblasts} \times \frac{adhered thymocytes}{total fibroblasts} \times 100$$

### Results

### 1—DM43-Coupled Affinity Chromatography

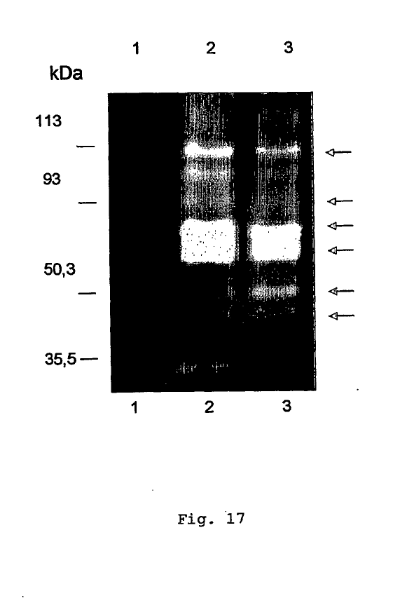

The application of breast and prostate adenocarcinomas supernatants (about 2 mL per run) to the DM43-coupled affinity column resulted in 2 protein peaks at 280 nm; the first eluted with the equilibrium buffer and the second with the glycine/HCl buffer, as indicated in FIGS. 16A and 16B. Volumes of 2.5 mL/tube were collected, which were precipitated with TCA for further analyses. The DM43-bound fractions (indicated by arrows in FIGS. 16A and 16B) were designated: MMDA (MDA MMPs) and MMCF7 (MCF7 MMPs).

### 2—Zymography

The analysis of MMDA and MMCF7 by zymography indicates the proteolytic activity of these samples, since all of them degraded the acrylamide-copolymerized gelatin. According to the technique, the hydrolysis area indicates the molecular mass of enzymes. As we can observe in FIG. 17, all the samples present intense degradation within the range of 50.3 and 113 kDa, where the active forms of MMPs 2 and 9 (=65 kDa) and the precursor forms of these MMPs (130, 92 and 72 kDa) are located. We also observe degradation slightly below 50.3 kDa, which may indicate the presence of the MMP-3 active form (45 kDa).

### 3—Imunorrevelation

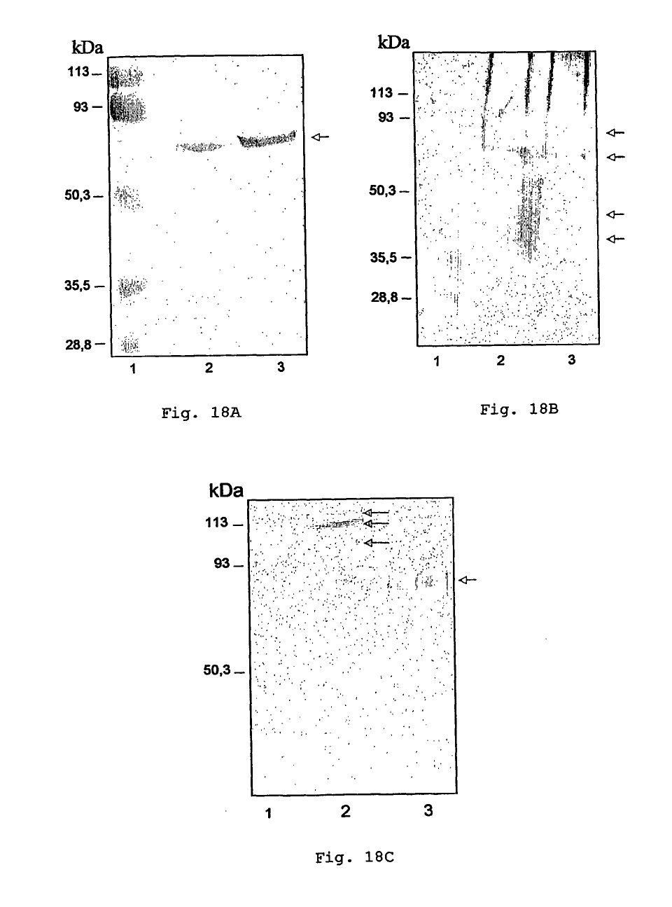

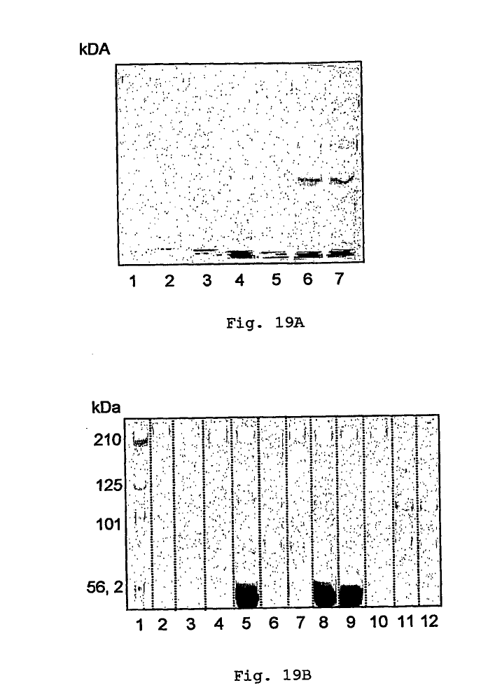

Following SDS-PAGE, all the samples (MMDA and MMCF7) were electrotransferred onto a PVDF membrane. The membranes were revelated separately with monoclonal antibodies (anti-MMP-2, 3 and 9) produced in mice. All the metalloproteinases were revealed with these 3 antibodies. MMP-2 was revealed in a band of ≅67 kDa (FIG. 18A). MMP-3 was evidenced in MMDA and MMCF7 in more than one protein band (≅90 and 70 kDa), in addition to the presence of the 67 kDa band. With minor intensity, a revelation of bands in the region ranging from 35.5 to 50.3 kDa was observed, which could correspond to the active form of MMP-3 (45 kDa) (FIG. 18B). MMP-9 was revealed in more than one protein band in MMDA and MMCF7 (=83, 95, 113 and another one of undetermined superior mass) (FIG. 18C)

### 4—Inhibition of Proteolytic Activity Using Fibronectin as Substrate

The proteolytic activity of MDA and MMCF7 was tested using fibronectin as substrate in SDS-PAGE. Although all the samples managed to degrade fibronectin chains, we verified a differential degradation among the samples. The extension of hydrolysis was evaluated with 0.5-15 μL of the tested materials. MMDA could degrade the fibronectin using 10 μL of material, while the degradation by MMCF7 was verified with 5 μL of the material (FIG. 19A).

For inhibition assays, we used the volume of 10 μL for both materials. When the samples were incubated with orthophenanthroline, both were inhibited, demonstrating the metalloproteasic nature of these samples. DM43 also showed to be effective in the inhibition of these metalloproteinases, as shown in FIG. 19B.

### 5—Cell Number Analysis

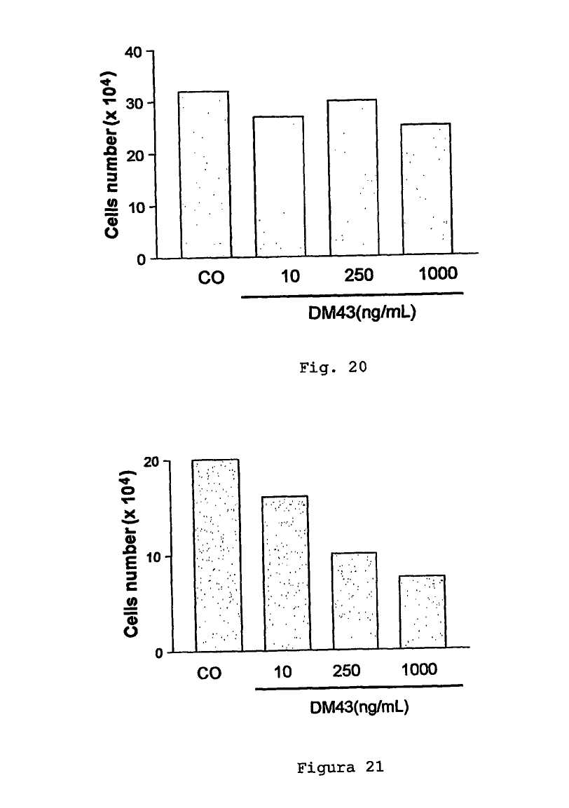

The results demonstrated a practically non-existent decrease in MDA cell line with any dose of the treatment (FIG. 20). In MCF7, the decrease is quite clear in a dose-dependent manner, i.e., the higher the DM43 dose, the lower the cellularity (FIG. 21). The bar chart represents 3 experiments developed with the tumor cell lines.

### 6—Cell Cycle Analysis

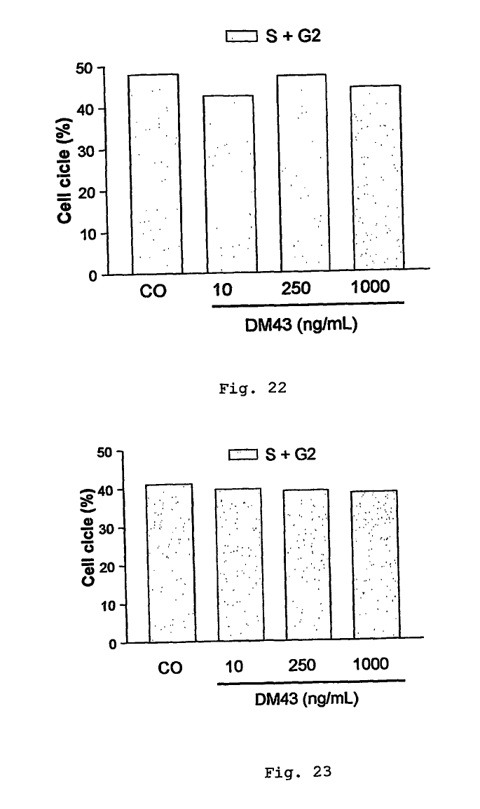

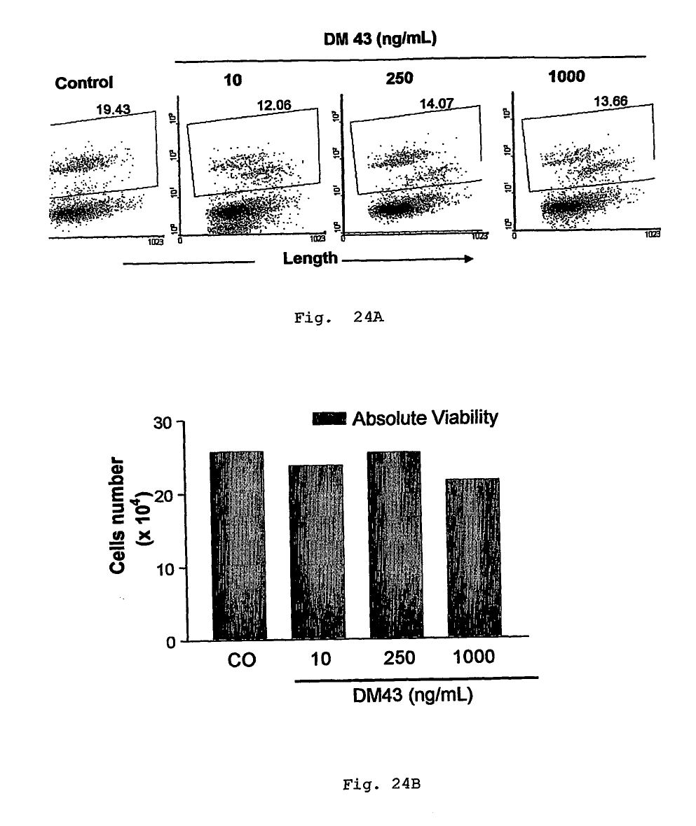

Cell cycle analysis of all assessed cell lines (breast and prostate adenocarcinomas) has not demonstrated any significant effect with any of the used doses of DM43 (FIGS. 22 and 23). The bar chart represents 3 experiments developed with the tumor cell lines.

### 7—Cell Death Analysis

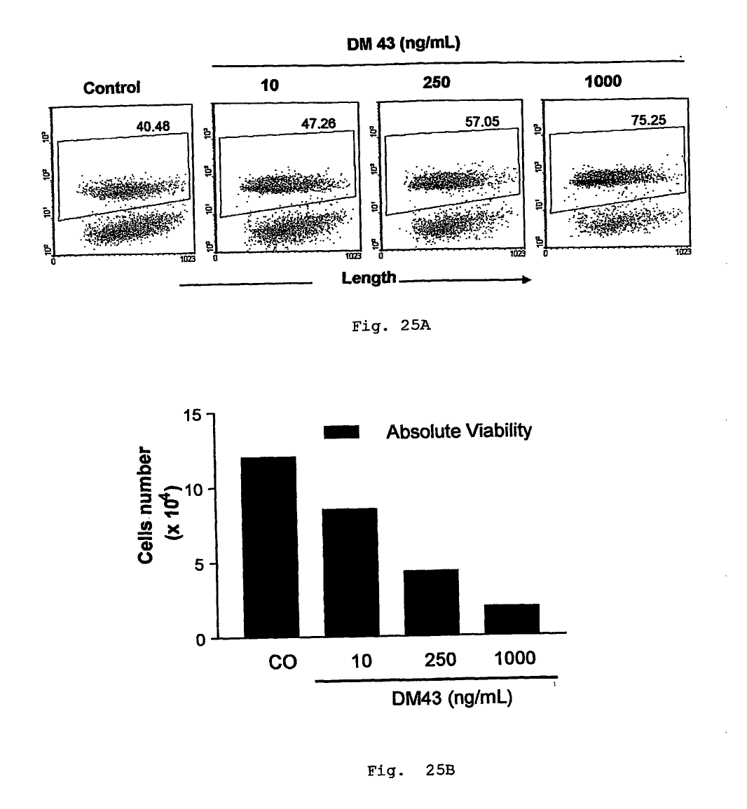

Cell death evaluated with DNA marker 7AA has not been evidenced in the MDA tumor cell line, as none of the DM43 doses could induce cell death (FIG. 24). As concerns MCF7 cell line, however, DM43 could induce death in a dose-dependent manner (1.2% with 10 ng/mL; 1.4% with 250 ng/mL and 1.9% with 1000 ng/mL of the treatment) (FIG. 25A). Using the absolute viability analysis, in which total cellularity (100%) was considered, reductions of 1.4%, 2.8% and 6.3% were verified in viable cells, respectively, in relation to the control with the 3 doses of DM43 treatment (FIG. 25B). The bar chart represents 3 experiments with the tumor cell lines.

### 8—Cell Adhesion Block

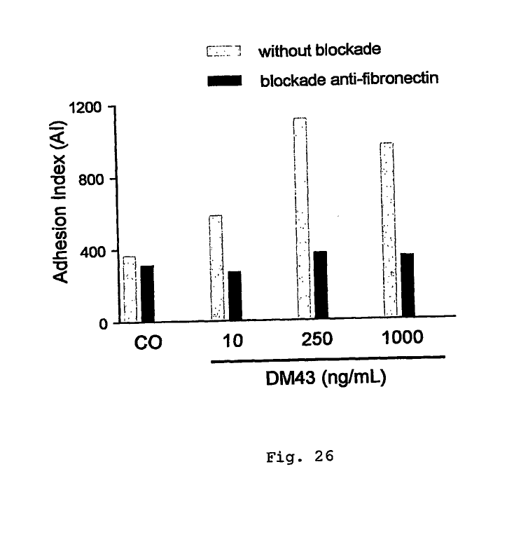

According to the adhesion index adopted, increases of 1.6, 3.1 and 2.7-fold were verified in relation to the control, respectively, using the DM43 treatment doses. The adhesion blockade using anti-fibronectin antibody before the addition of thymocytes resulted in decreased adhesion index to baseline values (FIG. 26).

Though several studies have been developed on the inhibition of MMPs as the main molecular target in diseases such as cancer, this inhibition efficacy still remains unclear. In fact, several drugs are under clinical trials, yet none of them have demonstrated to be totally effective so far.

Considering the existing homology between the metalloproteinases (SVMPs, MMPs and ADAMs) and the way by which they are inhibited (formation of noncovalent complexes, with stoichiometry of 1:1), the capacity of DM43, a potent SVMPs inhibitor, has been tested against the MMPs involved both in osteoarthritis and neoplasias, such as breast and prostate adenocarcinomas.

It is worth emphasizing that this is the first time that the inhibition capacity of DM43, a typical snake venom metalloproteinases inhibitor, has been demonstrated over another subfamily of metalloproteinases. The data presented here demonstrate the potentiality of DM43 in inhibiting MMPs, which are involved in diverse pathologies which do not have adequate therapies up to the moment.

### Pharmaceutical Compositions

According to the present invention, the pharmaceutical compositions consist of an effective amount of DM43 protein and a pharmaceutically acceptable vehicle.

The composition may be in the form of tablets, pills, capsules, or in the form of solutions or suspensions. Solid compositions contain the active ingredient mixed with non-toxic excipients which are appropriate for tablet manufacturing, such as starch, milk sugar, certain types of carbonates and/or bicarbonates, phosphates, etc. Tablets may be coated or not, depending on the spot within the gastrointestinal tract where drug disintegration and absorption should occur. In case of suspensions or aqueous solutions, excipients such as methylcellulose, sodium alginate, acacia gum, lecithin, etc. and one or more additives, such as preservatives, colorings, flavorings, thickening agents, etc. may be used.

An amount of DM43 protein shall be combined with the pharmaceutically acceptable vehicle so as to produce the appropriate dosage. In the present invention, pharmaceutical compositions may contain DM43 protein in preferential concentrations that range from 10 to 1000 ng/mL.

The dose for each patient shall depend on various factors, including the activity of the specific compound used, the age, body weight, general clinical picture, sex, [sic], diet, period and route of administration, excretion rate, combination with other drugs and the severity of the disease to be treated.

While the invention has been described in detail and with reference to specific examples thereof, it will be apparent to one skilled in the art that various changes and modifications can be made therein without departing from the spirit and scope thereof.

1. Pharmaceutical composition characterized by containing an effective amount of DM43 protein and a pharmaceutically acceptable vehicle.

2. Composition according to claim 1 characterized by effective concentrations of DM43 protein within the range of to 1000 ng/mL.

3. Method to inhibit metalloproteinases in human cells through the administration of an effective amount of DM43 protein.

4. Method for the treatment of tumor diseases in human patients, characterized by the administration of an effective amount of DM43 protein to the referred patient, as defined in claim 1.

5. Method for the treatment of breast tumors, prostate tumors, nape tumors, head tumors and lung tumors, using the composition of claim 1.

6. Method for the treatment of central nervous system diseases in human patients, using the composition of claim 1.

7. Method for the treatment of diseases such as multiple sclerosis and Alzheimer disease, using the composition of claim 1.

8. Method for the treatment of articular diseases such as osteoarthritis in human patients, using the composition of claim 1.

9. Use of DM43 protein in human treatment to inhibit the presence of matrix metalloproteinases.

10. Method for the treatment of tumor diseases in human patients, characterized by the administration of an effective amount of DM43 protein to the referred patient, as defined in claim 2.

11. Method for the treatment of breast tumors, prostate tumors, nape tumors, head tumors and lung tumors, using the composition of claim 2.

12. Method for the treatment of central nervous system diseases in human patients, using the composition of claim 2.

13. Method for the treatment of diseases such as multiple sclerosis and Alzheimer disease, using the composition of claim 2.

14. Method for the treatment of articular diseases such as osteoarthritis in human patients, using the composition of claim 2.
